(19) United States
(12) Patent Application Publication
(10) Pub. No.: US 2004/0132112 A1
(43) Pub. Date: Jul. 8, 2004

(54) Screening antibodies using an optical fiber array device capable of simultaneously performing multiple functional assays

(76) Inventor: Kimon Angelides, Houston, TX (US)

Correspondence Address: Eric P. Mirabel, Suite 100, 35 Technology Drive, Warren, NJ, 07059, US

(21) Appl. No.: 10/651,332

(22) Filed: Aug. 28, 2003

Related U.S. Application Data
(60) Provisional application No. 60/406,510, filed on Aug. 28, 2002.
(60) Provisional application No. 60/406,456, filed on Aug. 28, 2002.
(60) Provisional application No. 60/406,457, filed on Aug. 28, 2002.
(60) Provisional application No. 60/408,215, filed on Sep. 4, 2002.
(60) Provisional application No. 60/408,947, filed on Sep. 6, 2002.
(60) Provisional application No. 60/408,948, filed on Sep. 6, 2002.

(51) Int. Cl. G01N 33/53; G01N 33/567

(52) U.S. Cl. 435/7.2

(57) ABSTRACT

Disclosed is a method of screening binding molecules, including Monoclonal Antibodies, using an optical fiber array device. Target cells are coated onto a population of microbeads, wherein each microbead is coated with several cells of the same cellular type and has an assay and an assay reporter associated with it. Each of the cell-coated microbeads is positioned in a particular well formed in one end of a particular fiber in an array of optical fibers. The microbeads are contacted with binding molecules, and the results of an assay associated with a microbead are reported to the distal end of the array of fibers.

### PRIORITY CLAIM

Priority is hereby claimed to U.S. Provisional Application Serial Nos. 60/406,510; 60/406,456; 60/406,457 (all of which were filed on Aug. 28, 2002), to Serial No. 60/408,215, filed Sep. 4, 2002, and to Serial Nos. 60/408,947; 60/408,948, both filed on Sep. 6, 2002.

### BACKGROUND OF THE INVENTION

There are currently a number of different monoclonal antibodies approved for use in therapy by the United States Food & Drug Administration and other regulatory agencies. Monoclonal antibodies are deemed well-suited for therapy as they are specific for particular targets and do not bind to other cells or tissues, and because they can have any of a number of desired therapeutic effects, including cell-killing for tumor or infectious disease therapy.

Monoclonal antibodies are derived from a single clone of B-lymphocytes. These B cells are immortalized to provide a cell line able to indefinitely produce antibodies which are all specific to a particular target antigen.

In the conventional process for making monoclonal antibodies, a mouse is immunized with an antigen of interest, and its immune system is boosted with adjuvants so that it generates an enhanced response against the immunogen. The mouse B lymphocytes are extracted from the mouse spleens (which contain high numbers of B-lymphocytes), and then fused with an immortal myeloma cell line. Some of the hybridomas resulting from the fusion produce monoclonal antibodies to the antigen initially used to immunize the mouse. The single hybridoma (or hybridomas) among this sea of fused cells secreting antibody into the medium is then screened, and those with the desired characteristics are selected.

In view of the millions of different antibody-secreting cells in the hybridoma pool, selection is usually a labor-intensive trial and error process, the success of which is dependent on the screening method, the skill with which it is applied and the characteristics of the antibody sought. Conventional methods of screening involve one or more of enzyme linked immunosorbent assays (ELISA), Western blotting, dot blots and others. Oftentimes, no monoclonal antibodies targeting a particular antigen can be found using conventional methods, or, those that are found are of low affinity or specificity, or have undesirable and unanticipated effects for therapeutic use. Improved screening methods are clearly the key to success in continuing to find monoclonal antibodies effective in therapy.

Moreover, the need for intensive screening does not necessarily end when one isolates suitable mouse hybridoma candidates. Mouse-derived portions of a monoclonal antibody can cause immune reactions against the antibody upon human therapeutic use (called a human anti-mouse or “HAMA” response), especially when there is repeated dosing. This can lead to adverse patient consequences, in the worst case scenario, or, otherwise, a need for more frequent dosing or higher dosages of the antibody as the antibody is targeted by the patient's immune system and removed.

Several genetic engineering technologies have been developed to reduce the amount of mouse protein in an antibody and to make as much as possible human-derived. See, e.g., L. Riechmann et al., Nature, 1988; 332: 323-327; G. Winter, U.S. Pat. No. 5,225,539; C. Queen et al., U.S. Pat. No. 5,530,101. Deimmunised antibodies are antibodies in which the T and B cell epitopes have been eliminated using genetic engineering, as described in International Patent Application No. WO/9852976. They are designed to have reduced immunogenicity, when applied in vivo, and are also suitable for therapeutic use. Antibody fragments are smaller and therefore have less mouse protein than whole antibodies, and therefore, are likely to be less immunogenic. Antibody fragments include Fab, F(ab′)₂, and Fd fragments. These fragments can be isolated from antibody phage libraries generated using the techniques described in McCafferty et al., Nature 348:552-554 (1990), Clackson et al., Nature 352:624-628 (1991) and Marks et al., J. Mol. Biol. 222:581-597 (1991). Subsequent publications describe the production of high affinity (nM range) human antibodies by chain shuffling (Marks et al., Bio/Technology 10:779-783 (1992)), as well as combinatorial infection and in vivo recombination as a strategy for constructing very large phage libraries (Waterhouse et al., Nuc. Acids. Res. 21:2265-2266 (1993)). Single chain Fv molecules are another binding molecule which can be made using phage display techniques. See U.S. Pat. No. 5,565,332 and European Patent No. 0 589 877 B1. Again, they are smaller and have less mouse proteins than whole mouse antibodies.

Such genetic engineering technologies, however, requires additional screening steps. In humanized antibodies, the genes from mouse cells encoding the antigen-binding portions of the antibody (and as little as possible of the remainder) are re-combined with human genes encoding the remainder of the antibody. However, only some of the genes recombine properly to produce functional antibodies when expressed in a cell line. Phage display techniques involve random recombination of the human antibody gene repertoire. With either method, among the binding molecules that recombine properly, most will be low affinity, and the cell lines producing them may be unstable, low producing or prone to mutation so that, over time, the antibodies will assume different characteristics. Accordingly, use of humanization or related technology raises the need to do additional selection steps for stable cell lines producing properly “humanized” monoclonal antibodies with the desired characteristics.

Some companies (notably, Abgenix, Inc., Fremont, Calif., and Medarex, Inc., Annandale, N.J.) have genetically re-engineered mice themselves, so that the mice produce substantially human antibodies. Antibody-producing cells from such mice are then immortalized to make monoclonal antibodies. The resultant antibody is fully human in composition, although its properties, including specificity, may not or may not be adequate for therapy. Accordingly, as is true for the humanization methods, suitable candidates with the desired properties must be selected from a multitude of cells, and screening technology is a key to success.

One problem with either of the “mouse” approaches to making monoclonal antibodies is that the human immune response is not captured. Instead, one is limited to the mouse antibody and immune repertoire, and mice will often have a different antibody response to the same antigen than a human being. The phage display approach relies on random recombination of human antibody genes, and also does not capture the human immune response to an antigen. Accordingly, in an attempt to capture the human immune repertoire, researchers have attempted to isolate human peripheral B lymphocytes from human blood, and then immortalize them to make fully human monoclonal antibodies. This process requires extremely laborious and extended screening, and has been characterized by limited success. Humans cannot be immunized with antigens or “boosted” with adjuvants before collection of their lymphocytes, so the cells of interest, which produce antibodies to disease agents, are present in far smaller concentrations than when screening mouse cells extracted from immunized mice. Improved screening methods could allow one to select human antibody-producing cells, and make fully human monoclonal antibodies with desired characteristics.

In the conventional methods, the initial screening of antibody-producing cells is performed by placing individual cells into discrete wells of microtiter plates. This is accomplished with a limiting dilution of the population of antibody-producing cells, so that the probability is that there is only cell per well. One then screens each well in the plates with an assay of choice. Usually the assays used initially are for antibodies in the wells which bind to an antigen or target cell placed in the wells. Once the positive binders are located, their function or effects on cells or tissues are determined using so-called “functional” assays.

The various assays of the antibody-producing cells are, therefore, run sequentially rather than in parallel, slowing the process. Moreover, because screening is sequential, cells which fail to register as positives (including those which, for example, appear to fail to bind) in the earlier screens are rejected and not usually re-examined, because of the sheer numerousness of the pool of negatives. If the results of an early screen show some cell lines as false negative, the best therapeutic candidates can easily be overlooked.

U.S. Pat. No. 6,377,721 discusses a fiber optic array for use as a biosensor. Each fiber in the array has a well at one end, and each well is designed to accommodate one cell. The array is designed so that each cell is associated with an assay, and the outcome of the assays can be transmitted through the fiber and recorded at the opposite end of the fiber. In this manner, an array of data is generated, with each discrete point in the array representing a result from one particular assay. The device is designed for studying biologically active materials, in situ environmental monitoring, monitoring of bioprocesses and high throughput screening of large combinatorial chemical libraries. No mention or suggestion is made of using the device in screening of hybridomas or monoclonal antibodies, or in screening of compounds in the wells of microtiter plates.

A shortcoming of the device disclosed in U.S. Pat. No. 6,377,721 for screening antibody-producing cells is that with only one target cell per fiber well, that target cell may not come into contact with an antibody one is screening for, especially if the antibodies are present in low concentration. In addition, contact with and binding by one or only a few antibodies may not induce a detectable change in a particular target cell. It would be desirable to have a system which increased the likelihood of significant contact between target cells and antibodies, and effectively amplified the signal from a target cell bound by an antibody, over the “one cell per well” approach disclosed in U.S. Pat. No. 6,377,721. Also, the system should provide additional redundancy, so that more than one target cell/assay combination is presented to each of the antibodies in the assay plate wells. In this way, a failure to register by any one (or even several) of the assays will be less likely to register a false negative for the antibodies in the assay plate well where the failed assay occurred.

### SUMMARY OF THE INVENTION

The invention relates to an optical fiber array for determining and recording the results of essentially simultaneous assays performed on cells located at one end of the array, and its use in screening for binding molecules. Each fiber in the array has a well etched into one end of it. Each well is designed to contain within it a microbead. Each microbead is coated with cells. Responses of the cells on the microbeads in the assays are monitored by reporting them to the distal end of the fibers, and recording them there. The monitoring and reporting is accomplished with a reporter system which responds to light excitation, e.g., a fluorescence marker which fluoresces when illuminated by a laser. The fluorescence marker can either be a fluorescent dye loaded into the cells, a fluorescent dye coated on the microbeads and in contact with the cells, a reporter marker which responds by fluorescing when the cell responds to stimuli and expresses or secretes a particular protein, or other systems which can accomplish the same types of reporting. The fluorescence is detected at the distal end of the fibers and recorded, e.g., with a charge coupled device or similar optical recording device for detecting photons, which generates an array of data points, with each representing the results of one particular assay on one type of cell.

In a first embodiment, each microbead is coated with several cells which are all of the same type and all representative of a disease state. For example, all beads can be coated with tumor cells or infected cells. The cells of each bead (or each bead itself) are all associated with one particular assay, but different beads can be each associated with one of several different assays. Where different beads are associated with different assays, and each bead is at the end of one fiber in an array, the outcome of any particular assay can be separately recorded at the distal end of the fiber as a point in an array. It is therefore possible to assay a library of binding molecules, including antibodies, and record the effect discrete antibodies in the library have on the cells on discrete beads, as determined by several assays, each associated with one bead, which are all performed simultaneously.

The significant advantage of using beads coated with several cells is that the effect of an antibody in the assay associated with any particular bead is amplified. If one or more of the cells carried on a bead are affected by an antibody, this will be detected by the assay and reported by a fluorescence change and recorded. If one was using only one cell per fiber well, false negatives are more likely because of failure of the target antigen on the cell surface to come into contact with a targeting antibody; or, even if there is contact, antibody binding by only a few antibodies may fail to initiate a recognizable change in the cell due to differences in affinity of the antibodies, or differences in cell signaling functionality among different cells of the same type. Using several cells per bead provides amplification of signal and lessens the likelihood of false negatives.

An advantage of using a fiber array, is that each array can have a multitude of fibers (from 5,000 to 50,000 fibers per array can readily be achieved). Because the number of assays is more limited, more than one assay is likely to be associated with a particular array. This provides considerable redundancy for each of the assays, so that it is almost certain that more than one microbead/target cell/assay combination is presented to each of the assay plate wells. A failure to register by any one (or even several) of the assays will be less likely to be recorded as a false negative for the antibodies in the assay plate well where such failure occurred.

The devices discussed herein are well-suited for monitoring of antibody-producing cells which have been limit diluted and placed into the wells of a microtiter assay plate. The devices will preferably have a series of arrays designed such that one member in each array is aligned with each well in a microtiter plate, so as to allow simultaneously assaying and monitoring of one entire plate per pass. Alternatively, a single array, or several arrays, can be arranged in a pattern so that a multitude of fibers extend into each well in a multi-well assay plate. This can be accomplished by extending the ends of the fibers through a plate in the correct pattern to match the wells of the assay plate. The plate is then placed on top of the assay plate, so that the fiber ends enter the assay plate wells. Provided that the total number of assays associated with the array is substantially less than the number of fibers in each well, one can be assured that each assay will be carried out in each assay plate well, and there will be redundancy of assays.

The preferred assays include functional assays, which determine the effect that an antibody has on the function of a cell. The assays can be used to determine any of a number of cell function, including but not limited to: (i) cytotoxic activity toward cancerous cells; (ii) intra-cellular signaling, including G protein activation, phosphatidyl inositol signaling, or ion channel effects; (iii) Ca²⁺ regulation in live cells; (iv) effects on the JAK-STAT pathway (related to apoptosis); and (v) effects on tyrosine kinase activity, which is indicative of growth factor signaling. Simultaneously with a determination of function, assays can be included to determine antibody binding (a conventional enzyme-linked immunoadsorbant assay, “ELISA”), or to determine specificity, ie., that it binds only to the target cells and not to other cell or tissues.

If desired, one could also perform screenings of different types of cells, or different subpopulations of cells, using the device. One method to screen different cell types is by performing a sequential screening, first with one cell type coated on the beads, which are then assayed for antibody reactivity, light excited and the outcomes recorded; and then with another cell type on the beads, which are again assayed, excited and recorded. In the alternative, a microbead(s) can be coated with a plurality of different cell types, with each particular cell type encoded so that it can be identified in the array. Either of these arrangements of cells and microbeads allows assays for the effect of the antibodies being screened on different cell types to be performed and recorded in one pass-through.

Coating different cell types on the same microbead, or having more than one type of assay associated with each microbead, allows one to effectively multiply the number of assays which can be conducted by each array. The reporters for the assays can be selected to indicate the results of the different assays.

Coating different cell types on the same microbead, or coating different microbeads with different cell types, also allows one to perform differential screening allowing, for example, determination of a particular antibody's reactivity with both diseased and healthy cells, all in a rapid, high throughput manner. In one example, some beads could be coated with tumor cells and others with non-tumor cells of the same cellular type as the tumor cells. With such a system, one can simultaneously monitor the effect that an antibody has on the tumor cell and the healthy cell, and its specificity for tumor cells. The encoded bead/cell arrangement provides for an increase in throughput over sequential assaying of different cell types.

### BRIEF DESCRIPTION OF THE DRAWINGS

FIGS. 1A and 1B schematically depict the results, as seen on a CCD read-out, of four different simultaneous functional assays carried out with one cell type associated with each microbead. FIG. 1B depicts that some of the wells displayed fluorescence, indicating a positive assay result.

FIGS. 2A and 2B schematically depict the results, as seen on a CCD read-out, of four different simultaneous functional assays carried out with several cell types which are encoded so as to be identifiable, but with only one cell type associated with any particular microbead. FIG. 2B shows that several wells associated with each of the different microbeads displayed fluorescence.

FIG. 3 is a flow chart depicting making human hybridomas, screening them with an optical fiber device as described herein, and expressing the antibodies derived from the positive wells.

FIG. 4 is a side and partially cut-away view of a fiber array showing fiber wells and cell coated microbeads positioned in the fiber wells.

FIG. 5 schematically depicts operation of a fiber optic device in screening an assay plate and the results of assays recorded from such operation, where laser 41 passes excitation spectrum through a dichroic mirror 43, which passes signal to the arrays 31, and the signal is then passed back along the arrays and recorded and displayed as array 47.

### DETAILED DESCRIPTION OF THE INVENTION

One preferred use for the device described herein is for screening of monoclonal antibodies and monoclonal antibody-producing cells, although it can also be used for screening other binding molecules and products. As noted above, screening of antibody producing cells using simultaneous cell-based functional assays increases the likelihood of finding monoclonal antibodies suitable for therapeutic use. The device includes a number of arrays of optical fibers, and preferably, each of the arrays is aligned and positioned with respect to the other arrays so that the ends of each array will align with and be accommodated by one of the wells of a microtiter plate. Each array can include a multitude of fibers, for example, from 5,000 fibers to 50,000 fibers per array.

Each well in the microtiter plate would contain an expanded population of antibody-producing cells (B lymphocytes, transformed B lymphocytes or hybridomas). The ends of each of the fibers have a well formed therein, which accommodate the cell-coated microbead. Thereby, the microbeads are brought into contact with the antibodies in the microtiter plate wells. The assays associated with the cells and the microbeads cause the associated reporter to fluoresce. This results in an array of fluorescence (or of colors), with each point representing the results of an assay associated with one of the microbeads. Because in this embodiment of the device, each discrete fiber array is positioned in a microtiter plate well, assays are performed in an essentially simultaneous manner (of course, some assays may take longer than others to perform) on the antibodies in each well of the plate. This system is therefore ideally suited for use in high throughput screening.

This array can provide a wealth of information about the antibodies in the wells. Each array can include numerous fibers, each with a different microbead and assay, so that one is performing a number of simultaneous functional assays on the antibodies in each of the wells. This allows one to determine the effect the antibodies have on the cells associated with the fiber wells, with an immediate read-out and recordation of the results, providing a true high throughput functional screening.

In one design for the arrays, there are 96 fiber arrays arranged to align with the wells of a 96 well microtiter plate. This is a conventional plate used in screening of hybridomas or transformed B lymphocytes. Other alignments and designs for the arrays, designed for use with other types of wells or assay plates, can be readily made.

The size of the arrays will depend primarily on the attributes of the antibodies one is seeking to isolate for therapeutic use. Larger arrays can accommodate more assays, and are desirable to the extent that more cell-based based assays are needed (or even exist). Theoretically, fiber optic technology allows designing of each array so that it can accommodate up to millions of fibers and beads. The number of assays which can be performed by each array is therefore essentially unlimited.

(a) Fiber Optic Cables

Materials suitable for constructing fiber optic cables include, but are not limited to, glass and modified or functionalized glass, plastics (including acrylics, polystyrene and copolymers of styrene and other materials, polypropylene, polyethylene, polybutylene, polyurethanes, Teflon™), polysaccharides, nylon or nitrocellulose, resins, silica or silica-based materials including silicon and modified silicon, carbon, metals, inorganic glasses, plastics, and a variety of other transparent or translucent polymers. The preferred materials allow optical detection and do not themselves appreciably fluoresce.

(b) Attaching Microbeads to Fiber Wells

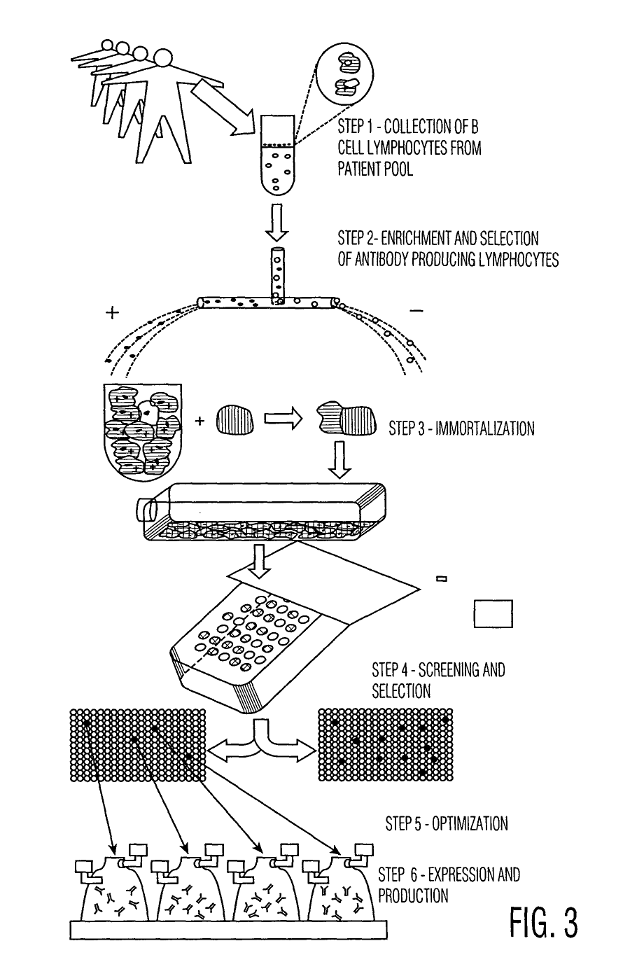

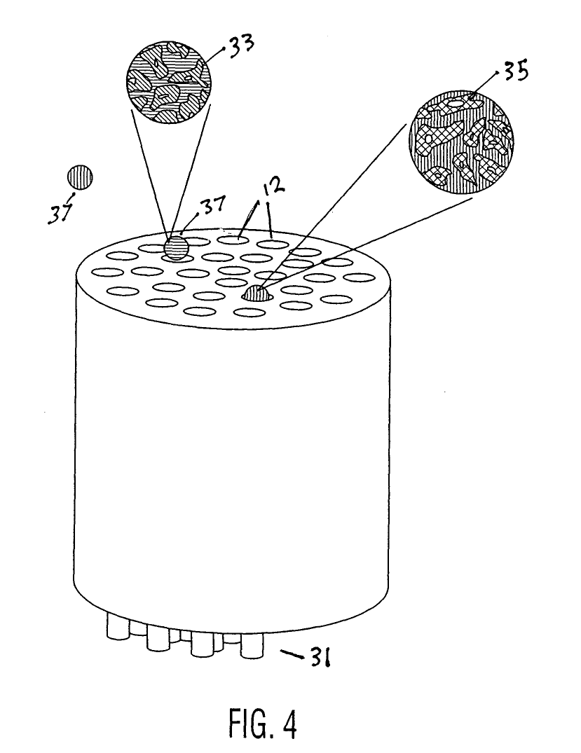

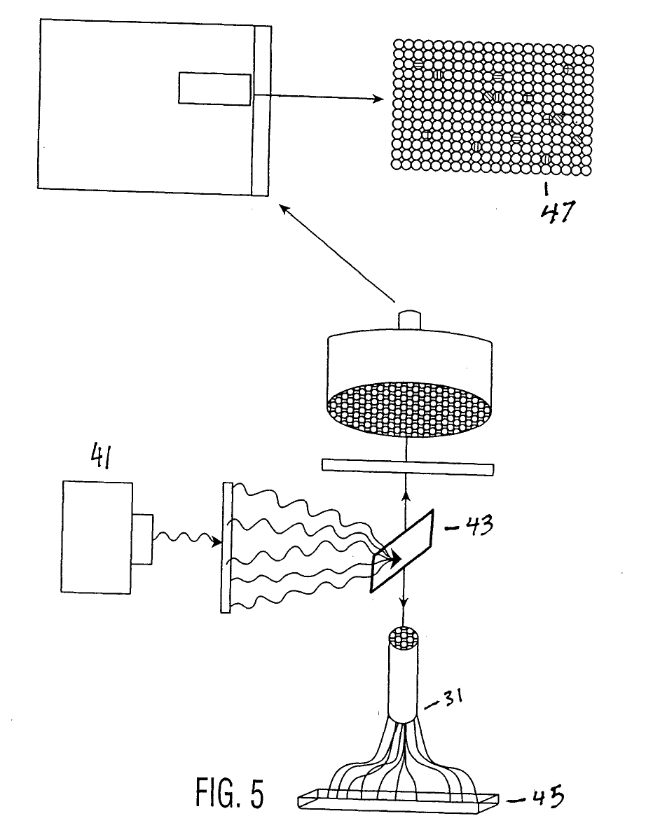

A fiber optic array 31 is depicted in FIG. 4, and a number of such arrays 31 are shown in FIG. 5. FIG. 4 shows individual fibers co-axially disposed and joined along their lengths. At one end of each of the fibers, are wells 12 which can each accommodate a cell coated microbead 33 or 35. The microbeads can be of any suitable biologically compatible material, and can range in size from between 2 to 1,000 microns. The microbeads are maintained in the wells 12 by covalent attachment with chemically or biologically altered or active sites, including sites with a functional group added, electrostatically altered sites, hydrophobically or hydrophilically functionalized sites, or other well-known means which interact with either the cells or the beads. The sites are preferably arranged in a high enough density to effectively capture and hold a cell-coated microbead, when the array is inverted and placed into a well of a microtiter plate 45. The microbeads can also be “loaded” into the optical fiber wells through associations and coating of the wells with poly-L-lysine, collagen, extracellular matrix components, or even biocompatible glues such as poly-L-lactic acid (see also FIG. 3).

Methods of covalent attachment include, but are not limited to: (i) the addition of a pattern of chemical functional groups including amino groups, carboxy groups, oxo groups and thiol groups, that can be used to attach to the cells or the microbeads, and wherein the cells or microbeads also generally include corresponding reactive functional groups on their surfaces; (ii) the addition of a pattern of adhesive that can be used to bind to the cells or the microbeads; (iii) the addition of a pattern of charged groups for the electrostatic attachment of the cells or microbeads, where the cells or microbeads include oppositely charged groups; (iv) the addition of a pattern of functional groups that renders the wells differentially hydrophobic or hydrophilic, such that the addition of similarly hydrophobic or hydrophilic cells or microbeads under suitable conditions will result in association of the cells and microbeads to the wells on the basis of hydroaffinity.

Alternatively, the microbeads can be attached to the wells using biological binding partner pairs, including, but not limited to, antigen/antibody pairs, enzyme/substrate or inhibitor pairs, receptor-ligand pairs, carbohydrates and their binding partners (including lectins and others). Alternatively, the interior surfaces of the fiber wells may be coated with a thin film or passivation layer of biologically compatible material, including, but not limited to: fibronectin, any number of known polymers including collagen, polylysine and other polyamino acids, polyethylene glycol and polystyrene, growth factors, hormones, or cytokines. Similarly, binding ligands as outlined above may be coated onto the surface of the wells. In addition, coatings or films of metals such as gold, platinum or palladium may be employed. The microbeads can also be non-covalently associated with the fibers in the wells. For example, a physical barrier may be used, i.e., a film or membrane over the microbeads in the wells.

(c) Making Fiber Wells

The wells 12 in FIG. 4 in the ends of the fibers can be formed using any of a variety of well-known techniques, including, but not limited to, photolithography, stamping techniques, pressing, casting, molding, microetching, electrolytic deposition, chemical or physical vapor deposition employing masks or templates, electrochemical machining, laser machining or ablation, electron beam machining or ablation, and conventional machining. The technique used will depend on the composition and shape of the fiber. The depth of the wells will depend on the size of the microbeads to be added to the wells.

One method of creating wells in the ends of the fibers is by using a selective etching process which takes advantage of the difference in etch rates between core and cladding materials. This process has been previously disclosed by Pantano, et al., Chem. Mater. 8:2832 (1996), and Walt, et al., in U.S. Pat. No. 6,023,540, incorporated by reference. The etch reaction time and conditions are adjusted to achieve control over the resultant microwell size and volume. Microwells can thus be sized to accommodate microbeads of different sizes.

(d) Coating Cells on Microbeads

The microbeads suitable for use in the invention can be any type which can be coated with cells, and can in turn be adhered to the wells in the fibers. Coating cells on microbeads is described in U.S. Pat. No. 5,653,922, which relates to porous cross-linked polymeric microbead produced by suspension polymerization of a high internal phase emulsion. These microbeads can directly attach cells by including them in cell growth media, or be modified to improve cell attachment, with, for example, a variety of bridging molecules, including antibodies, lectins, glutaraldehyde, and poly-L-lysine. In addition, sulfonation of microbeads, as described using the processes and reagents in U.S. Pat. No. 5,653,922, can increase cell attachment rate. Other methods of coating include use of fibronectin, collagen, Matrigel-1™, or extracellular matrix components.

(e) Assay Systems and Reporters

As discussed above, screening of antibodies is most efficient and effective when done with functional assays in a high throughput manner. A functional assay for screening for antibodies effective against cancer cells focuses on cytotoxic activity to cancerous cells. Methods to assay antibody cytotoxic effect include loading the cells one is interested in killing (including infected cells or tumor cells) with a fluorescent dye, such as propidium iodide or EthD-1, which enters cells with damaged membranes and undergoes a 40-fold enhancement of fluorescence upon binding to nucleic acids, thereby producing a bright red fluorescence in dead cells (ex/em.about.495 nm/.about.635 nm). EthD-1 is excluded by the intact plasma membrane of live cells.

Another suitable marker is Almar Blue, which fluoresces if the cell is active. One can also use the LIVE/DEAD.RTM. Viability/Cytotoxicity Assay Kit (L-3224) by Molecular Probes, Inc. of Eugene, Oreg., utilizing a two color reporter system. The cell-permeant esterase substrate calcein AM is nonfluorescent until converted by enzymatic activity to highly fluorescent calcein, which is retained within live cells and imparts an intense green fluorescence. Ethidium homodimer-1 undergoes a fluorescence enhancement upon binding nucleic acids, producing a bright red fluorescence. This dye is excluded from cells that have intact plasma membranes but is readily able to enter dead cells. Thus, live cells fluoresce green, while dead cells fluoresce red.

Other fluorescent reporters can indicate if the antibody affects other cell functions, for example, intra-cellular signaling. Fluo-3 can indicate changes in the cell surface receptors that end up in calcium signals, which can indicate that the antibody is affecting one or more of G protein activation, phosphatidyl inositol signaling, or ion channels. Phosphatidyl inositol signaling can be indicated by phosphodiesterase substrates, including several unique fluorescent phosphatidyl inositol derivatives.

Molecular Probes has available several reagents for studying Ca²⁺ regulation in live cells. Fluorescent nucleotides, including analogs of ATP, ADP, AMPPNP, GTP, GDP, GTP-γ-S and GMPPNP can be used, and the GTP analogs may be particularly useful in the assay of G-protein-coupled receptors. Protein complementation assays of the JAK-STAT pathway (related to apoptosis) can be reported by a protein-protein interaction, using the reconstitution of catalytic activity of βgalactosidase, dihydrofolate reductase, or any such enzyme that is able to cleave or form a bond of a substrate, fluorescent or otherwise. Tyrosine kinase activity can also be measured by this method which is indicative of growth factor signaling.

These fluorescent dyes are added to the cells by, for example, incubating the cells with the dye. The cells may be rinsed to wash excess dye from the outer surface of the cells. The cells are then adhered to the microbeads as described above.

(f) Identifying and Screening of Different Types or Subpopulations of Target Cells

Instead of having all target cells of the same type coated on the beads, it is possible to have different types or sub-populations of target cells coated on different beads, and then identified according to their position in the array. This could be an advantage when determining antibody specificity, i.e., one could have the target cell (tumor, infected or other) on certain beads, and other cell and tissue types on other microbeads. The different cell types are encoded and coated onto microbeads. The beads can be randomly mixed prior to attachment to fiber wells. The encoding allows the microbeads with a particular cell type to be identified in the array.

Cells may be encoded with a single fluorophore or chromophore dye, or with ratios of such dyes. Alternatively, cells may be encoded by either injecting a non-toxic fluorescing compound into the cell cytoplasm or by employing natural or genetically-engineered cells lines which exhibit chemiluminescence or bioluminescence, such as green fluorescent protein mutants. Although a plurality of cell populations may be randomly mixed, the identity and location of each cell type is determined via a characteristic optical response signature when the array is illuminated by excitation light energy. Either a single fluorophoric or chromophoric material or dye can be used for encoding the cells, or, in the alternative, two or more encoding materials or dyes may be used to encode different cell populations.

A wide variety of fluorophores, chromophores, stains or a dye compounds may be used for encoding cells. Encoding dyes may either permeate or not permeate the cell membrane. Non-permeating dyes may be conjugated with acetoxymethyl ester to allow them to be taken up by cells. Conventional conjugate or reactive cell membrane stains, cell tracers, or cell probes such as fluoresceins, rhodamines, eosins naphthalimides, phycobiliproteins, and nitrobenzoxadiazole may be utilized. In other embodiments, cyanine dyes, such as SYTO® (Molecular Probes), amine-reactive dyes, thiol-reactive dyes, lipopilic dyes, and DNA intercalators, such as acridine orange, may be employed. Alternatively, fluorogenic or chromogenic enzyme substrates may be taken up by the cells, processesed by intracellular enzymes, such as glycosidases, phosphatases, luciferase, or chloramphenicol acetyltransferase, and provide encoding for cell populations. Cell organelle dye probes or cell membrane probes such as carbocyanines and lipophilicaminostyrls may also be employed for encoding.

Tables 1 and 2 of U.S. Pat. No. 6,377,721 list various types of dyes and their corresponding excitation and emission wavelengths which are suitable for encoding cell populations in sensor arrays of the present invention. In addition, a particularly useful reference for selecting other types of encoding dyes is R. P. Haugland, Handbook of Fluorescent Probes and Research Chemicals (6^{th} ed.), Molecular Probes Inc. (Eugene, Oreg., 1996).

The fluorophores, chromophores, stains and dyes discussed above can be monitored using a conventional fluorescence microscope, as is used for monitoring of the fluorescence activity of the assay reporting systems associated with the cells. If needed, the same laser which excites the fluorescence reporter on the cells and provide assay results, can induce fluorescence of the encoding material. Such systems of lasers and microscopes, and the use of them, are well known in the art, and conventionally used with fluorescence markers to excite them, by supplying the correct excitation frequency.

The following examples illustrate the use of the invention in antibody screening. It should be noted that the same types of techniques described above and in the examples can be used to screen for other binding molecules, including proteins, having the desired characteristics.

### EXAMPLE I

### Screening with One Cell Type

After generating a series of hybridoma cell lines, they are plated into 96 well microtiter plates, with each well likely to contain only one hybridoma cell (following a limiting dilution of hybridoma cells). In the alternative, it is possible to perform only a partial limit dilution, or even no dilution, so that there is likely to be more than one hybridoma cells in each microtiter plate well. The methods described herein can still isolate the wells with reactive antibodies, and the hybridomas from such wells can then be isolated and further limit diluted and screened to find the candidates of interest.

An embodiment of the optical fiber device having 96 arrays, each of which aligns with one well in the microtiter plate, is employed for screening. The hybridoma cells were generated either by: (i) immunizing mice with tumor cells and then performing a conventional fusion, following collection of lymphocytes from their spleens, or (ii) fusing peripheral B lymphocytes extracted from human patients who all express a particular tumor. The same tumor cell line which was used to immunize the mice (or which the patients were exposed to) is adsorbed onto microbeads using the techniques described above. The microbeads are then treated so as to be able to provide results of one of several different assays, including one or more assays for cytotoxicity. The assays include fluorescent reporter systems which can be monitored by a fluorescent microscope following laser excitation. The microbeads are then adsorbed into fiber wells.

The ends of the fibers are now placed into the microtiter plate wells, and allowed to react with the antibodies generated by the hybridomas therein. Each plate will generate 96 fluorescent arrays (with multiple points in each array, each point representing one microbead), when excited with the laser. The assay results are monitored through the microscope and recorded by a charge coupled device (CCD) array. See FIG. 5.

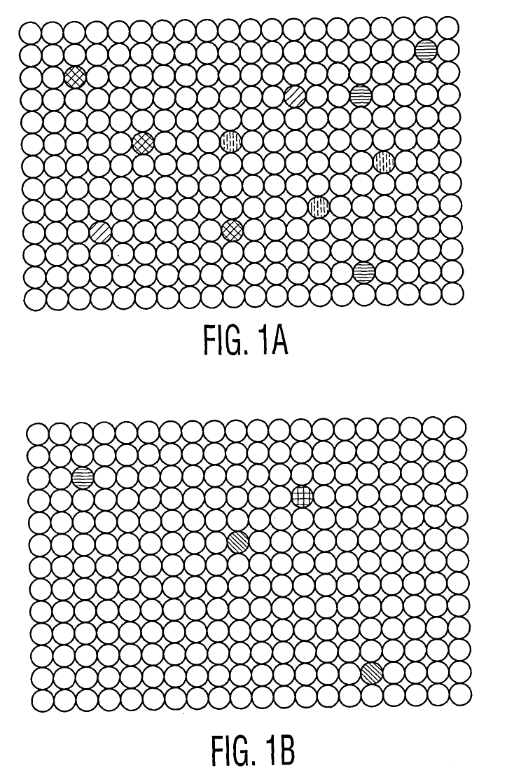

FIG. 1 schematically shows the results for a group of arrays placed in the wells of a microtiter plate well where there is one cell type coated on the microbeads. It can be seen in the second frame that certain points in the array, reflecting the assay results for certain microbeads, have changed color, indicating a positive assay for the antibodies in the corresponding microtiter plate well.

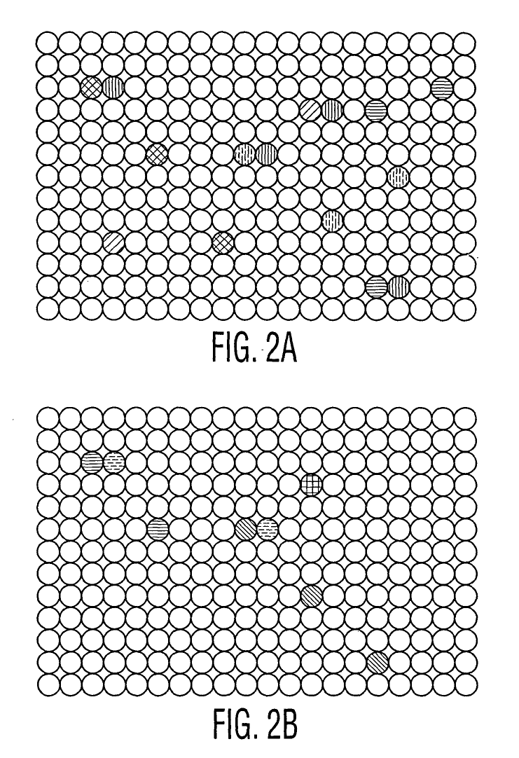

FIG. 5 depicts the device and the detection process schematically, with arrays 10 extending into the wells of microtiter plate 17. Laser 21 passes excitation spectrum through a dichroic mirror 22, which passes signal to the arrays 10, and the signal is then passed back along the arrays and recorded and displayed as read-out 23. This step is repeated for each of the 96 well microtiter plates which contain hybridomas. A rich series of data is thereby generated in a high throughput manner (see FIGS. 1 and 2), as the assays are run in each of the microtiter plates in a simultaneous manner.

### EXAMPLE II

### Screening with Two Cell Types

Hybridomas are generated and plated onto series of 96 well microtiter plates as described in Example I. In this case, however, two cell lines are adsorbed onto two different sets of color coded microbeads: (i) the same tumor cell line which was used to immunize the mice (or which the patients were exposed to) (ii) a non-tumor cell line, which is preferably the non-mutated counterpart of the tumor cell line. The two different sets of microbeads are separately encoded with a color or fluorescence marker. As described in Example I, the cells on each set of microbeads are then treated so as to report the results from one of several different assays, which results can be monitored by a fluorescent microscope following laser excitation. One or more of the assays is for cytotoxicity. The microbeads are then adsorbed into the wells in the fiber wells in the device.

The ends of the fibers are now placed into the microtiter plate wells, and allowed to react with the antibodies generated by the hybridomas therein. If using a 96 well plate, each plate will generate 96 fluorescent arrays (with multiple points in each array), when excited with the laser. This step is repeated for each of the 96 well microtiter plates which contain hybridomas. Data is generated which displays the results of the assays for both the tumor and non-tumor cell lines (see FIG. 2) for one array in one microtiter plate well. The encoding allows one to determine whether the antibodies which kill the tumor cells also kill the non-tumor cells. Other cell lines could also be encoded and adsorbed to microbeads and assayed, if one was attempting to further define the specificity of the antibodies.

FIG. 2 schematically shows results from a single array in a single microtiter plate well, and the use of four assays. Some of the microbeads are encoded (indicated by an adjacent red marker), indicating a different cell type associated with the tagged microbeads. The second frame shows that some of both the tagged and untagged microbeads were positive (indicated by a changed color) after excitation. This means that the assays associated with the microbeads which changed color were positive for some of both types of cells on the microbeads, and thus the antibodies in the assay plate well affect both types of cells. These antibodies are therefore non-specific and not desired candidates.

### EXAMPLE III

### Procedures Following Isolation of Desired Antibody-Producing Cells

Using the techniques in the examples and otherwise described herein, simultaneous assaying and recording of a number of properties of the antibodies within the 96 microtiter plate wells is provided. The cells in the wells which are determined to contain antibodies best suited for therapy are then extracted from the well, grown, subcloned and humanized or affinity matured, or otherwise optimized (if desired). Fully human hybridomas derived from human lymphocytes may not need any further manipulation, except perhaps subcloning to find high expressing, stable cell lines suitable for production. The same techniques described in Examples 1 and 2 may then be used on the subcloned, humanized or optimized cell lines to screen for suitable antibody-producing cell lines, produced following such steps. These subsequent screenings can also be performed in a high throughput manner, with a number of functional assays, and other assays, if desired, performed essentially simultaneously. Use of more assays will ultimately provide for improved screening and selection of the best candidates. Such additional assays can be readily determined by those skilled in the art, depending on the therapeutic target, the patient population, the nature of the target cells, and other factors readily apparent.

Following the foregoing screenings with functional assays, the putative candidates can be further screened for other properties, including but not limited to whether they bind to target cells, their affinity for the target cells, their stability and lack of immunogenicity, and others, all apparent to those skilled in the art, and dependent on factors associated with therapeutic products. Because these screenings will be performed on only a few candidates which have cleared the functional assay screenings performed using the optical fiber device described herein, there is no significant loss in throughput rate, even if these additional steps are performed in sequence rather than in parallel.

Defined Term; claim Scope

It should be understood that the terms and expressions used herein are exemplary only and not limiting, and that the scope of the invention is defined only in the claims which follow, and includes all equivalents of such claims. The term “Monoclonal Antibodies” as used in the claims refers to all monoclonal antibodies and derivatives and fragments thereof having binding activity, including but not limited to mouse, humanized, human, and Deimmunised antibodies, and fragments including Fab, F(ab′)₂, and Fd fragments, and single chain Fv binding molecules.

What is claimed is:

1. A method of screening binding molecules, including Monoclonal Antibodies, comprising:
coating target cells onto a population of microbeads, wherein each microbead is coated with several cells of the same cellular type and has an assay and an assay reporter associated with it;
positioning each of the cell-coated microbeads in a particular well formed in one end of a particular fiber in an array of optical fibers;
contacting the microbeads with binding molecules; and
reporting the results of an assay associated with a microbead to the distal end of the array of fibers.

2. The method of claim 1 further including recording the results of the reported assay.

3. The method of claim 1 further including illuminating the microbeads with a laser to induce a change in fluorescence of the reporter.

4. The method of claim 3 wherein the recording of results is performed using a charge coupled device.

5. The method of claim 4 wherein the binding molecule is a protein and the microbeads are contacted with protein-producing cells.

6. The method of claim 5 wherein the cells which induced or mediated particular changes in fluorescence in particular reporters associated with particular assays are isolated and grown.

7. A method of screening Monoclonal Antibodies comprising:
coating target cells onto a population of microbeads, wherein each microbead is coated with several cells of the same cellular type and has an assay and an assay reporter associated with it;
positioning each of the cell-coated microbeads in a particular well formed in one end of a particular fiber in an array of optical fibers;
placing a series of antibody-producing cells producing Monoclonal Antibodies into a series of assay plate wells;
positioning the end of the array with the microbeads into an assay plate well; and
reporting the results of the assays associated with microbeads to the distal end of the array of fibers.

8. The method of claim 7 wherein there are a plurality of arrays of optical fibers positioned in relation to each other so that they align with each of the assay plate wells.

9. The method of claim 7 wherein the Monoclonal Antibodies are placed in the wells of the assay plate in a manner such that it is likely that there is only one antibody-producing cell per well.

10. The method of claim 7 further including illuminating the microbeads with a laser to induce a change in fluorescence of the reporter.

11. The method of claim 10 wherein the results of the assays are recorded using a charge coupled device.

12. The device of claim 10 wherein wherein the data generated and recorded by the charge coupled device is in the form of any array of fluorescent points, with each point in the array representing one of the microbeads, and wherein a change in fluorescence of a microbead following its placement in the assay plate well represents that the cells associated with that microbead were affected or bound by the Monoclonal Antibodies in the assay plate well.

13. The method of claim 12 wherein the antibody-producing cells which produced those Monoclonal Antibodies which induced or mediated particular changes in fluorescence in particular reporters associated with particular assays are isolated and grown.

14. The method of claim 7 wherein the assay reporters report one or more of: (i) cytotoxic activity toward cancerous or infected cells; (ii) intra-cellular signaling, including G protein activation, phosphatidyl inositol signaling, or ion channel effects; (iii) Ca²⁺ regulation in live cells; (iv) effects on the JAK-STAT pathway; (v) effects on tyrosine kinase activity; and (vi) antibody binding.

15. The method of claim 7 wherein the cells coated on the microbeads are tumor cells, or cells infected with a virus, bacteria, prion, parasite or other pathogen.

16. A method of screening Monoclonal Antibodies comprising:
coating target cells onto a population of microbeads, wherein each microbead is coated with several cells of the same cellular type that has an assay and an assay reporter associated with it, but there are two or more subpopulations of microbeads each coated with one of a plurality of cellular types, and each subpopulation is encoded to indicate which cellular type it is coated with;
positioning each of the cell-coated microbeads in a particular well formed in one end of a particular fiber in an array of optical fibers;
placing a series of antibody-producing cells producing Monoclonal Antibodies into a series of assay plate wells; and
positioning the end of the array with the microbeads into an assay plate well; and
reporting the results of the assays associated with microbeads to the distal end of the array of fibers.

17. The method of claim 16 wherein the Monoclonal Antibodies are placed in the wells of the assay plate in a manner such that it is likely that there is likely to be only one antibody-producing cell per well.

18. The method of claim 16 further including illuminating the microbeads with a laser to induce a change in fluorescence of the reporter.

19. The method of claim 16 wherein the results of the assays are recorded using a charge coupled device.

20. The method of claim 16 wherein the data generated and recorded by the charge coupled device is in the form of any array of fluorescent points, with each point in the array representing one of the microbeads, and wherein a change in fluorescence of a microbead following its placement in the assay plate well represents that the cells associated with that microbead were affected or bound by the Monoclonal Antibodies in the assay plate well.

21. The method of claim 20 wherein the assay reporters report one or more of: (i) cytotoxic activity toward cancerous or infected cells; (ii) intra-cellular signaling, including G protein activation, phosphatidyl inositol signaling, or ion channel effects; (iii) Ca²⁺ regulation in live cells; (iv) effects on the JAK-STAT pathway; (v) effects on tyrosine kinase activity; and (vi) antibody binding.

22. The method of claim 20 wherein the cells coated on the microbeads are tumor cells, or cells infected with a virus, bacteria, prion, parasite or other pathogen.

23. The method of claim 22 wherein the antibody-producing cells which produced those Monoclonal Antibodies which induced or mediated particular changes in fluorescence in particular reporters associated with particular assays in microbeads coated with one cellular type, but did not induce fluorescence changes in microbeads coated with other cellular types, are isolated and grown.

24. A method of screening Monoclonal Antibodies comprising:
coating target cells onto a population of microbeads, wherein each microbead is coated with several cells of two or more different cellular types, wherein each cellular type has an assay and an assay reporter associated with it, and each cellular type is encoded to indicate which cellular type it is;
positioning each of the cell-coated microbeads in a particular well formed in one end of a particular fiber in an array of optical fibers;
placing a series of antibody-producing cells producing Monoclonal Antibodies into a series of assay plate wells; and
positioning the end of the array with the microbeads into an assay plate well; and
reporting the results of the assays associated with microbeads to the distal end of the array of fibers.

25. The method of claim 24 wherein the cells coated on the microbeads are tumor cells, or cells infected with a virus, bacteria, prion, parasite or other pathogen.

26. The method of claim 25 wherein the antibody-producing cells which produced those Monoclonal Antibodies which induced or mediated particular changes in fluorescence in particular reporters associated with particular assays in one cellular type, but did not induce fluorescence changes in other cellular types, are isolated and grown.
